(19) United States
(12) Patent Application Publication
(10) Pub. No.: US 2006/0093592 A1
(43) Pub. Date: May 4, 2006

(54) Synbiotics

(75) Inventors: Rukmini Cheruvanky, Folsom, CA (US); Reddy Sastry Cherukuri, Folsom, CA (US); R. Erick Pecha, Folsom, CA (US); Eliot Drell, El Dorado Hills, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NutraCea, El Dorado Hills, CA (US)

(21) Appl. No.: 11/243,361

(22) Filed: Oct. 3, 2005

Related U.S. Application Data
(60) Provisional application No. 60/615,908, filed on Oct. 4, 2004.

(51) Int. Cl. A61K 35/74; A61K 36/899; A61K 31/715; A61K 31/663; A61K 31/56; A61K 31/355

(52) U.S. Cl. 424/93.45; 424/750; 514/54; 514/102; 514/171; 514/458; 424/195.16

(57) ABSTRACT

A novel product comprising a mixture of probiotic and prebiotic ingredients for the treatment of IBS, IBD, Crohn's disease, antibiotic induced diarrhea and other bowel disorders is described herein. Stabilized rice bran derivatives, including stabilized rice bran, RiSolubles, RiceMucil, and Cea100, are used as the source of prebiotics. The prebiotic source is not only rich in fructo-oligosaccharides, but also has potent antioxidants and phytonutrients for intestinal health and the proliferation of bifido-bacteria in the intestines. The probiotics used are different combinations and concentrations of Lactobacilli species, depending on the specific gastrointestinal disease which is targeted. When refrigerated, the products are shelf-stable (95-99%) for at least one year.

### RELATED APPLICATIONS

This application claims the benefit of U.S. patent application Ser. No. 60/615,908, filed Oct. 4, 2004, entitled “SynBiotics,” incorporated by reference herein, in its entirety and for all purposes.

### BACKGROUND

The gastrointestinal tract is diverse in its physiological, biochemical and molecular structure and function. It is therefore no surprise that disease states have a highly varied etiology according to the organ of involvement, such as ulcerative colitis, inflammatory bowel disease (IBD) and irritable bowel syndrome (IBS). The direct effects of these diseases or malnutritive states can result in a loss of mucosal immunity and integrity, microflora degradation, or even ulceration of the gastrointestinal (GI) tract.

Therefore, the role of any medicinal or immunotherapeutic intervention must be to counter-balance the loss of optimum gut function. Many of the current medicinal approaches are imperfect for improving gastrointestinal health. Gut conditions from indigestion to ulcerative colitis are treated with a gamut of pharmaceutical drugs. Most cases in the gastrointestinal wards of the hospital relate to gastro-esophageal reflux, peptic ulceration, non-ulcer dyspepsia, constipation, IBD, IBS and Crohn's disease. Celiac disease, caused by a gluten allergy, affects 0.5 to 1% of the U.S. population. The vast majority of patients with gut disorders do not have underlying cancer but this condition is important to exclude.

Treating IBD is a major challenge to gastroenterologists. Fibers are known to help bowel moments and may relieve the symptoms of IBS. Metamucil™ and Citrucel™ are the fiber of choice for gastroenterologists. These increasing formulations, however, suffer from many side effects including allergies, gas formation, flatulence, and abdominal distention. Recently a pharmaceutical product named Lotronex™ was marketed for IBS but patients given this drug suffered serious side effects.

For many gastrointestinal conditions, the current therapies offered are imperfect. A great demand therefore exists for novel pharmaceutical and “natural” approaches to treating these conditions.

### BRIEF SUMMARY OF THE INVENTION

The present invention provides novel mixtures of probiotics and prebiotics, as well as methods for administering the mixtures, which are useful for the effective treatment of several GI disorders, without unwanted side effects. “SynBiotics” is the term used for a composition which comprises probiotics in addition to prebiotics with natural antioxidants.

In one embodiment, the invention provides a synbiotic composition, wherein the composition comprises (i) a probiotic composition, wherein the probiotic composition comprises a Lactobacillus species; and (ii) a prebiotic mixture, wherein the prebiotic mixture comprises at least one stabilized rice bran derivative, wherein the stabilized rice bran derivative comprises at least one natural antioxidant selected from the group consisting of a tocol, a phytosterol, γ-oryzanol and inositol hexaphosphate (IP6). In a related embodiment, the stabilized rice bran derivative is selected from the group consisting of a water-soluble fraction of stabilized rice bran, a water-insoluble fraction of stabilized rice bran, and a combination of water-soluble and water-insoluble fractions of stabilized rice bran.

In a related embodiment, the prebiotic mixture of the synbiotic composition comprises at least one component selected from the group consisting of an oligosaccharide, a polysaccharide, and a fructo-oligosaccharide. In another related embodiment, the prebiotic mixture further comprises a phytonutrient. In yet another related embodiment, the prebiotic composition is hypoallergenic. In yet another related embodiment, the amount of said prebiotic is between 5% and 60% w/w of the synbiotic composition.

In another embodiment of the synbiotic composition of the invention, the probiotic component of the composition additionally comprises a Bifidobacteria species. In yet another embodiment, the Lactobacillus species of the probiotic component comprises at least one species selected from the group of species consisting of L. caesi, L. acidophilus, L. plantarum, and L. rhamnosus. In yet another embodiment, the probiotic composition further comprises a yeast species, e.g., a Saccharomyces species such as Saccharomyces boulardii.

In yet another embodiment, the invention provides a synbiotic composition in a dosage form containing 10⁶ to 10¹⁰ viable colony forming units per dose. In yet another embodiment, the synbiotic composition comprises less than 20% prebiotics on a weight-percentage basis. In a related embodiment, at least 40% w/w of the synbiotic composition of the invention consists of a yeast species such as Saccharomyces boulardii. In yet another embodiment, the synbiotic composition comprises at least 20% or at least 40% prebiotics on a weight-percentage basis.

The invention additionally provides an embodiment in which the novel synbiotic compositions are encapsulated in a vegetable capsule. In a related embodiment, the vegetable capsule is enterically coated. In another embodiment, the synbiotic composition of the invention is a liquid at room temperature. In other embodiments, the synbiotic composition is in tablet or powder form. In yet another embodiment, the synbiotic composition is stable for at least one year when stored at temperatures between approximately 0 and 10 degrees Celsius.

The invention additionally provides methods for treating or alleviating the symptoms of various gastrointestinal ailments by administering the novel synbiotic compositions described herein. Specifically, in one embodiment, the invention provides a method for treating or preventing irritable bowel syndrome (IBS) and related bowel disorders, comprising the step of administering a therapeutically effective dose of the synbiotic compositions to a patient in need of such treatment. In a related embodiment, the patient has been diagnosed with an illness selected from the group consisting of: inflammatory bowel syndrome, Crohn's disease, ulcerative colitis, indeterminatal colitis, microscopic colitis, collagenous colitis, idiopathic inflammation of the small bowel, Clostridium difficile diarrhea, travelers' diarrhea, and antibiotic-induced diarrhea. In a related embodiment, the method of treatment enhances said subject's immune function, improves the subject's gut health, induces production of epithelial enzymes, induces the synthesis of vitamins in the intestines of the subject, results in a substantial reduction in the levels of toxins in the subject's GI tract, induces apoptosis of cancer and precancerous cells in the subject, improves the overall gastrointestinal and colonic health of the subject, reduces bloating, abdominal distention or gas production, improves bowel regularity, prevents harmful microbial or viral infections or alleviates the symptoms thereof, or facilitates healthy weight loss in the subject. In a related embodiment, the method of treatment achieves more than one of the aforementioned beneficial health effects.

In yet another embodiment, the invention provides a method for carrying out a home study to determine the efficacy of a synbiotic composition, comprising the steps of: providing an online questionnaire to candidate patients; receiving a completed questionnaire from the candidate patients electronically; determining from the completed questionnaire whether a candidate patient is eligible for the home study; providing an eligible candidate with a test synbiotic formulation, instructions for administering the test synbiotic formulation over a predetermined time period, and a second questionnaire for the eligible candidate to self-record the effects of the synbiotic administration; collecting the self-recorded data from the second questionnaire after the predetermined time period has passed; and

evaluating the self-recorded data. In a related embodiment, the test synbiotic formulation comprises (i) a probiotic composition, wherein the probiotic composition comprises a Lactobacillus species; and (ii) a prebiotic mixture, wherein the prebiotic mixture comprises at least one stabilized rice bran derivative, wherein the stabilized rice bran derivative comprises at least one natural antioxidant selected from the group consisting of a tocol, a phytosterol, γ-oryzanol and inositol hexaphosphate (IP6)

### BRIEF DESCRIPTION OF THE DRAWINGS

FIG. 1 shows the stability of SynBiotics 1, SynBiotics 2, and SynBiotics 3 encapsulated formulations over a period of 360 days/one year (FIG. 1A). FIG. 1B shows a graph of a similar experiment analyzing SynBiotics 3 stability.

FIG. 2 shows a flow-chart describing a system for conducting home trials for determining the efficacy of one or more SynBiotic formulations.

### DETAILED DESCRIPTION

The present invention provides formulations of prebiotics and probiotics in predetermined ratios, as well as capsules comprising the formulations for treating various gastrointestinal ailments. In certain aspects, the formulations of the present invention are mixtures comprising novel combinations of selected probiotics and prebiotics that work together to provide beneficial effects.

In certain embodiments, the compounds used in the formulations and methods of this invention include stabilized rice bran derivatives, which can include, but are not limited to, rice bran oil, an enzyme-treated stabilized rice bran, a solubilized fraction of an enzyme-treated stabilized rice bran, or mixtures thereof. Preferably, the stabilized rice bran derivative utilized is the solubilized fraction. Other compounds used in formulations and methods of this invention include fortification agents which can include, but are not limited to, a glucosamine derivative, methylsulfonylmethane, yucca concentrate, grape seed extract, curcumin, ginger powder, boswellin, and ashwagandha. The compounds of the invention can also comprise an extract of active ingredients of rice bran derivatives, such as tocols.

The term “tocol” refers to E complex vitamins known as tocopherols and tocotrienols which have antioxidant properties. There are at least ten different isomeric forms of these vitamins. The term “tocol composition” refers to any composition comprising tocols.

The terms “phytonutrient” and “phytochemical” are used interchangeably to describe plant-derived terpenes, carotenoids, limonoids, and phytosterols with properties beneficial to human health.

As used herein, the term “enzyme treated stabilized rice bran derivative” refers to an enzyme-treated stabilized rice bran made by mixing a stabilized rice bran with an aqueous solution in a 15% to about a 35% aqueous slurry w/w; adding an enzyme to the aqueous rice bran slurry to convert starch to dextrin, and then directly drying the dextrin solution to form an enzyme treated stabilized rice bran derivative. The enzyme treated stabilized rice bran comprises about 20% to about 30% total dietary fiber.

The processing of rice bran and the nutritional composition of rice bran, as well as other aspects of the stabilized rice bran derivatives used in formulations of this invention, are further described in issued U.S. Pat. Nos. 6,126,943 and 6,350,473, entitled “Method for Treating Hypercholesterolemia, Hyperlipidemia, and Atherosclerosis,” both of which are incorporated herein by reference.

As used herein the term “stabilized rice bran derivative solubilized fraction” refers to a fraction obtained during a partitioning process. Specifically, after a stabilized rice bran aqueous slurry is enzymatically treated, it can be pumped into a centrifuge where the insoluble fraction precipitates out of the aqueous solution. The aqueous material is then pumped to a dryer and then dried. This dried aqueous portion produces the soluble fraction. The constituents of such a soluble fraction (e.g., RiSolubles™, a product of NutraCea, Inc.) are listed in Table 1, below, and a method of preparation of the stabilized rice bran derivative solubilized fraction is described in detail in Example 1, herein.

TABLE 1
RiSolubles ™
INGREDIENTS:

Stabilized Rice Bran and Germ, non-chemically predigested and
separated from insoluble fiber.

GUARANTEED SPECIFICATIONS:

| | | | |
|---|---|---|---|
| Protein | 7-12% | Ash | 3-7% |
| Fat | 25-32% | Moisture | 2-7% |
| Total Carbohydrates | 50-60% | Free Fatty Acids | <3% |
| Total Dietary Fiber | 0-6% | | |

MICROBIOLOGICAL:

| | | | |
|---|---|---|---|
| Total Plate Count | Maximum | 10,000 | CFU/g. |
| Total Coliform | Maximum | 100 | CFU/g. |
| E. coli | Maximum | <10 | CFU/g. |
| Salmonella | Negative | | |
| Yeast | Maximum | 100 | CFU/g. |
| Mold | Maximum | 100 | CFU/g. |

PHYSICAL:

| | |
|---|---|
| Appearance | Fine Powder |
| Color | Pale Yellow |
| Flavor | Sweet, Nutty |
| Bulk Density (G/Cc) | 0.31 |

ANALYTICAL DATA
MACRONUTRIENTS (g/100 g)

| | |
|---|---|
| Protein (N × 6.25) | 7.50 |
| Fat | 26.50 |
| Saturated Fatty Acid | 4.80 |
| Total Carbohydrate | 57.50 |
| Available Carbohydrate | 54.50 |
| Ash | 5.00 |
| Moisture (100 degree vac.) | 3.00 |
| Crude Fiber | 4.60 |
| Total Dietary Fiber | 3.00 |
| Soluble Fiber | 3.00 |
| Calories/100 g. | 486.50 |

VITAMINS

| Vitamin A; Carotenoids (meg/100 g) | |
|---|---|
| β-Carotene | 8.10 |
| α-Carotene | 0.00 |
| Lycopene | 0.20 |
| Lutein | 26.10 |
| Zeaxanthin | 10.90 |
| Precryptoxanthin/Cryptoxanthin | 1.27 |
| Total Carotenoids | 46.57 |
| Vitamin B Complex (mg/100 g) | |
| Vitamin B1 | 3.60 |
| Vitamin B2 | 0.46 |
| Vitamin B3 | 76.60 |
| Vitamin B5 | 5.82 |
| Vitamin B6 | 5.81 |
| Vitamin B12 (mcg/100 g) | <0.500 |
| Vitamin C(mg/100 g) | <0.500 |
| Vitamin E Complex (mg/100 g) | |
| Tocopherols (T) | 8.00 |
| Tocotrienols (T3) | 10.00 |
| Total Tocols (T + T3) | 18.00 |
| Other Micronutrients (mg/100 g) | |
| Folic Acid (mcg/100 g) | 36.17 |
| Biotin (mcg/100 g) | 14.70 |
| Choline | 150.00 |
| Inositol | 1490.0 |
| γ-Oryzanol | 248.10 |
| Phytosterols (mg/100 g) | |
| β-Sitosterol | 211.90 |
| Stigmasterol | 68.69 |
| Campesterol | 117.32 |
| Brassicasterol | 15.25 |
| Total Phytosterols | 413.16 |

MINERALS (mg/100 g)

| | |
|---|---|
| Sodium | 15.75 |
| Potassium | 1562.00 |
| Calcium | 8.30 |
| Magnesium | 170.80 |
| Phosphorous | 763.00 |
| Manganese | 3.20 |
| Iron | 1.90 |
| Copper | 0.07 |
| Zinc | 1.75 |
| Chromium (ppm) | <1 ppm |
| Total Sugars(g/100 g) | 13.83 |
| (No Lactose) | |

Rice bran derivatives have been shown to have more than a hundred (100) potent anti-oxidants including vitamin E and its isomers (tocopherols (T) and tocotrienols (T₃)), collectively referred to as tocols. A tocol-rich substance is a mixture containing one or more compounds selected from tocopherols (T), tocotrienols, and tocotrienol-like (T₃-like) compounds. Stabilized rice bran is the highest natural source of vitamin E.

Additional antioxidants in stabilized rice bran derivatives include, but are not limited to, γ-oryzanol, β-carotene, several known flavanoids, phytosterols, lipoic acid, ferulic acid and inositol hexaphospate (i.e., “IP6”). Some of these compounds are present in stabilized rice bran derivatives at concentrations which are much higher than in any of the known natural sources of the compounds. Ferulic acid, for example, is a phytochemical found in seeds of plants such as in brown rice, whole wheat and oats, as well as in coffee, apple, artichoke, peanut, orange and pineapple. Ferulic acid protects our cells form ultraviolet rays and neutralizes reactive oxygen species in the body, thereby preventing the reactive oxygen species from causing damage to our DNA. Being an antioxidant, it also reduces the level of cholesterol and triglyceride in the body and thus lowers the risk of heart diseases. IP6 is a phosphorylated form of inositol commonly found in fiber-rich plant foods. IP6 is hydrolyzed by phytase enzymes in the digestive tract to yield inositol. IP6 supports a cell's natural defense against damaging hydroxyl free radicals by chelating with reactive iron. In combination with probiotics, antioxidants provide exceptional additional defense and increase the immune system's ability to resist invasive pathogens associated with gastrointestinal disorders.

The term “probiotics,” as used herein refers to naturally-occurring “friendly” bacteria, e.g., lactobacillus and/or bifidobacteria species. These bacteria are an integral part of everyone's digestive system. Bifidobacteria are gram-positive anaerobes. They are non-motile, non-spore forming and catalase-negative. They have various shapes, including short, curved rods, club-shaped rods and bifurcated Y-shaped rods. Their name is derived from the observation that they often exist in a Y-shaped or bifid form. The guanine and cytosine content of their DNA is between 54 mol % and 67mol %. They are saccharolytic organisms that produce acetic and lactic acids without generation of CO₂, except during degradation of gluconate. Examples of bifidobacteria species include Bifidobacterium adolescentis, Bifidobacterium bifidum, Bifidobacterium animalis, Bifidobacterium thermophilum, Bifidobacterium breve, Bifidobacterium longum, Bifidobacterium infantis and Bifidobacterium lactis. Specific strains of bifidobacteria useful as probiotics include Bifidobacterium breve strain Yakult, Bifidobacterium breve RO7O, Bifidobacterium lactis Bb12, Bifidobacterium longum RO23, Bifidobacterium bifidum RO71, Bifidobacterium infantis RO33, Bifidobacterium longum BB536 and Bifidobacterium longum SBT-2928. A preferred bifidobacterium species for use in the SynBiotics of the present invention is Bifidobacterium longum.

Lactobacilli are gram-positive facultative anaerobes which normally inhabit the human intestine and vagina. They are non-spore forming and non-flagellated rod or coccobacilli. They are either aerotolerant or anaerobic and strictly fermentative. In the homofermentative case, glucose is fermented predominantly to lactic acid. Lactobacilli are also classified as lactic acid bacteria (LAB). To date, 56 species of the genus Lactobacillus have been identified. Lactobacilli used as probiotics include Lactobacillus acidophilus, Lactobacillus brevis, Lactobacillus bulgaricus, Lactobacillus casei, Lactobacillus cellobiosus, Lactobacillus crispatus, Lactobacillus curvatus, Lactobacillus fermentum, Lactobacillus GG (Lactobacillus rhamnosus or Lactobacillus casei subspecies rhamnosus), Lactobacillus gasseri, Lactobacillus johnsonii, Lactobacillus plantarum and Lactobacillus salivarus. Lactobacillus plantarum 299v strain originates from sour dough. Lactobacillus plantarum itself is of human origin. Other probiotic strains of Lactobacillus are Lactobacillus acidophilus BG2FO4, Lactobacillus acidophilus INT-9, Lactobacillus plantarum ST31, Lactobacillus reuteri, Lactobacillus johnsonii LA1, Lactobacillus acidophilus NCFB 1748, Lactobacillus casei Shirota, Lactobacillus acidophilus NCFM, Lactobacillus acidophilus DDS-1, Lactobacillus delbrueckii subspecies delbrueckii, Lactobacillus delbrueckii subspecies bulgaricus type 2038, Lactobacillus acidophilus SBT-2062, Lactobacillus brevis, Lactobacillus salivarius UCC 118 and Lactobacillus paracasei subsp paracasei F19. Preferred species of Lactobacillus include L. caesi, L. acidophilus, L. plantarum, and L. rhamnosus.

Other probiotic microbes can also be used in the SynBiotic compositions of the present invention. For example, the gram-positive facultative anaerobe Streptococcus thermophilus can be used. Enterococcus faecium SF68 is a probiotic strain that has been used in the management of diarrheal illnesses. The yeast Saccharomyces boulardii has been used to treat diarrhea associated with antibiotic use.

When consumed as food or as dietary supplements, probiotics can enhance health in several ways. They can stimulate the immune system, eradicate harmful and toxigenic bacteria and viruses, help with food and nutrient assimilation and promote gastro-intestinal and colon health. Many individuals are depleted in bifidobacteria in their digestive system and consequently suffer from gastrointestinal problems.

Probiotics are discussed generally in the following patent applications, hereby incorporated by reference in their entirety for all purposes: U.S. Patent Application Publication No. 2004-0072794 A1 (Nutritional formulations containing synbiotic substances); U.S. Patent Application Publication No. 2004-0067223 A1 (Probiotic compositions for the treatment of inflammatory bowel disease); U.S. Pat. No. 6,942,857 (Microorganisms for preventing and/or treating obesity or diabetes mellitus); PCT Application Publication No. WO01/93904 (Method of treating gastrointestinal diseases associated with species of genus Clostridium); European Patent Application Publication No. 1 384 483 A1 (Probiotics for treatment of irritable bowel disease (IBS) through improvement of gut neuromuscular function); PCT Application Publication No. WO 99/17788 (Composition of treatment of candidiasis); PCT Application Publication No. WO 04/014403 (Microorganisms for inhibiting obesity and diabetes mellitus); U.S. Pat. No. 6,641,808 (Composition for treatment of obesity); and U.S. Patent Application Publication No. 2003-0147857 A1 (Probiotic/prebiotic composition and delivery method).

“Prebiotics” are compositions which serve, at least in part, as a source of food for friendly bacteria. Prebiotics thus facilitate the proliferation of probiotic organisms in the intestines. Fructo-oligosaccharides are a preferred food of bifidobacteria. Stabilized rice bran and water soluble fractions thereof (e.g., RiSolubles™) are especially rich in fructo-oligosaccharides. In addition, stabilized rice bran derivative soluble fractions typically comprise polysaccharides and oligosaccharides with potent antioxidants (see Table 1).

SynBiotics™ is a trade name used by the NutraCea company (El Dorado Hills, Calif.) for a variety of therapeutic compositions containing appropriate combinations of probiotics and prebiotics with potent antioxidants. In preferred embodiments, a SynBiotic composition comprises a mixture of beneficial microbes and a stabilized rice bran derivative in a form that is suitable for oral ingestion. For example, a probiotic culture and prebiotics may be formulated as a mixture and encapsulated. Such a capsule preferably includes an enteric coating when one or more of the probiotic organisms in the encapsulated formulation is non-viable under acidic conditions (e.g., the acidic conditions of the stomach). Regardless of whether the formulation is in liquid, capsule, powder or tablet form, a shelf-life of at least a year, or more, is desirable.

Typical SynBiotic formulations of the present invention include one or more Lactobacillus species as part of the probiotic component. Preferred species of Lactobacillus include L. caesi, L. acidophilus, L. plantarum, and L. rhamnosus. The Lactobacillus component of the probiotic portion of a particular SynBiotic formulation may be augmented by the inclusion of one or more additional microbial species, including additional Lactobacilus species, a Bifidobacteria species, and/or a species of yeast such as Saccharomyces. A preferred species of Bifidobacteria is Bifidobacteria longum. If more than one bacterial species is present, the species may be present in differing amounts or equal amounts, on a weight percentage basis, with respect to each other. For example, a SynBiotic formulation can comprise 50% probiotic on a weight percentage basis, i.e., a 500 mg dose of a SynBiotic formulation can comprise 250 mg of a probiotic composition. The probiotic composition, in turn, can consist of 30% Lactobacillus plantarum, 20% Lactobacillus rhamnosus, and 50% Bifidobacterium longum.

The inclusion of particular proportions of a yeast species in SynBiotics formulations increases their efficacy still further, particularly against antibiotic-associated colitis caused by Clostridium difficile. Members of the genus Clostridium are Gram-positive, spore-forming anaerobic rods. The bacterial spores tolerate extreme conditions in which other bacteria cannot survive. In their active form, these bacteria secrete powerful exotoxins that are responsible for such diseases as tetanus, botulism and gas gangrene.

Antibiotics are ubiquitous among children and adults for bacterial infections (e.g., traveler's diarrhea) and sometimes they are prescribed for viral infections, including HIV. Antibiotic-mediated disruption of the normal flora can lead to fungal infections, such as invasive candidiasis, or antibiotic-associated colitis caused by Clostridium difficile.

For example, for treating colitis and other illnesses, e.g., traveler's diarrhea, that are associated with antibiotic treatment, SynBiotic formulations are preferred which include, per dose, on a weight percentage basis, as much or more of a yeast species as the prebiotic component (e.g., RiSolubles). For example, a SynBiotic formulation that includes 10% yeast and 10% prebiotic is preferred. Even more preferred are SynBiotic formulations that include greater percentages of yeast than prebiotic. For example, a formulation that includes 10% prebiotics may include 10%, 20%, 30%, 40% or 50% yeast on a weight percentage basis. Formulations that include 10% prebiotics and 50% yeast are especially preferred. A preferred yeast species is Saccharomyces boulardii.

“Inflammatory bowel disease” (IBD) is a collective term involving, chronic inflammatory disorders of the gastrointestinal tract such as ulcerative colitis, Crohn's disease and associated bowel disorders which result in serious consequences. The symptoms range from abdominal pain, cramping, diarrhea, rectal/intestinal bleeding, weight loss and fever. These symptoms may be progressive with repeated severe relapses. IBD has no cure. The choice of treatment consists of anti-inflammatory and immunosuppressive drugs and, as a last resort, surgery.

The SynBiotic formulations of the present invention supply a more specific bacterial equilibrium to the GI tract. This will alter the bacterial balance in the GI tract and substantially ameliorate or even cure IBD.

“Irritable bowel syndrome” (IBS) is a condition affecting approximately 10 to 20% of the global population and is characterized by chronic abdominal pain and altered bowel habits. The condition occurs most frequently in women and usually begins in those between 20 and 30 years of age. It is one of the most common disorders encountered in any gastrointestinal practice. It is associated with abdominal distention, painful cramps, a sense of incomplete evacuation as well as an overall uncomfortable feeling.

IBS arises mainly due to a disturbance in the large intestines muscular movement (motility); there is no abnormality in the intestinal structure. Predisposing factors may be a low residue diet, emotional stress, bowel consciousness, and laxatives abuse.

Changes in diet may help alleviate symptoms in some patients, but no diet is applicable to all patients. Increasing dietary fiber and eliminating gastrointestinal stimulants such as caffeine containing beverages may be beneficial. Other possible treatment may include: anxiety reducing measures, such as regular exercise; the administration of anticholinergic medications before meals; and counseling in cases of severe anxiety or depression.

SynBiotics-1™, SynBiotics-2™ and SynBiotics-3™ are examples of preferred embodiments of SynBiotic dietary supplements comprising particular combinations and ratios of probiotic microorganisms, together with a specified amount of the prebiotic, RiSolubles. RiSolubles is rich is fructo-oligosaccharides, on which friendly bacteria thrive. The formulations in SynBiotics-1, -2, and -3 are encapsulated. Each formulation is described in detail, below.

Synbiotics-1™ is a novel, specially selected probiotic with RiSolubles, a stabilized rice bran derivative, as the prebiotic (see Table 2). It is delivered as an enterically coated vegetable capsule containing RiSolubles blended with a specially selected bacterial combination of Bifidobacterium longum, Lactobacillus rhamnosus and Lactobacillus plantarum, suitable for the relieving the symptoms of irritable bowel syndrome (IBS). In one embodiment, the blend consists of about 2.75 billion colonies for unit (cfu)/capsule. A typical treatment regimen is one capsule, taken two times daily, but the dosage and regimen can be altered according to the patient's needs.

TABLE 2
SYNBIOTICS-1 FORMULA CAPSULES

| Ingredients | Percent | mg/capsule |
|---|---|---|
| Neb. Cultures* Probiotic Mix 1 | 50% | 250 mg |
| 30% Bifidobacterium longum | | |
| 30% Lactobacillus plantarum | | |
| 40% Lactobacillus rhamnosus A | | |
| RiSolubles ™ | 50% | 250 mg |
| Total: | 100% | 500 mg |

Total CFU/capsule 2.75 billion CFU
Other ingredients: Maltodextrin, magnesium stearate, hydroxypropylmethyl cellulose, methacrylic acid, methyl methacrylate copolymer.
*Nebraska Cultures (Walnut Creek, California)

SynBiotics-2™ is a novel, specially selected probiotic with RiSolubles, a stabilized rice bran derivative, as the prebiotic (see Table 2). It is delivered as an enterically coated vegetable capsule containing RiSolubles blended with a mixture of Bifidobacterium longum, Lactobacillus rhamnosus and Lactobacillus plantarum, suitable for the relieving the symptoms of irritable bowel syndrome (IBS). In one embodiment, the blend consists of about 3.0 billion colony forming unit (cfu)/capsule. A typical treatment regimen is one capsule, taken two or three times daily, preferably thirty minutes after meals, but the dosage and regimen can be altered according to the patient's needs. SynBiotics-2 provides a more potent dose of colony forming bacteria relative to SynBiotics-1 per capsule, with a slightly increased proportion of Bifidobacterium.

TABLE 3
SYNBIOTICS-2 FORMULA CAPSULES

| Ingredients | Percent | mg/capsule |
|---|---|---|
| Neb. Cultures* Probiotic Mix 2 | 50% | 250 mg |
| 50% Bifidobacterium longum | | |
| 30% Lactobacillus plantarum | | |
| 20% Lactobacillus rhamnosus A | | |
| RiSolubles ™ | 50% | 250 mg |
| Total: | 100% | 500 mg |

Total CFU/capsule 3.0 billion CFU
Other ingredients: Maltodextrin, magnesium stearate, hydroxypropylmethyl cellulose, methacrylic acid, methyl methacrylate copolymer.
*Nebraska Cultures (Walnut Creek, California)

Synbiotics-3™ is an enterically coated vegetable capsule containing RiSolubles blended with a specially selected bacterial combination (Bifidobacterium longum, Lactobacillus rhamnosus, Lactobacillus plantarum) and further comprising a yeast (Saccharomyces bolardii), where the combination of the probiotic with prebiotic is engineered to suppress the growth of Clostridium difficile and alleviate the colitis which accompanies antibiotic-induced diarrhea. A typical treatment regimen is one capsule, taken two or three times daily, preferably thirty minutes after meals, but the dosage and regimen may be altered according to the patient's needs.

TABLE 4
SYNBIOTICS-3 FORMULA CAPSULES

| Ingredients | Percent | mg/cap |
|---|---|---|
| Neb. Cultures* Probiotic Mix 3 | 40% | 200 mg |
| 33.3% Bifidobacterium longum | | |
| 33.3% Lactobacillus plantarum | | |
| 33.3% Lactobacillus rhamnosus A | | |
| Saccharomyces boulardii | 50% | 250 mg |
| RiSolubles ™ | 10% | 50 mg |
| Total: | 100% | 500 mg |

Total CFU/capsule 3 billion CFU Nebraska Cultures Mix, 5 billion
CFU Saccharomyces boulardii
Other ingredients: Maltodextrin, magnesium stearate, hydroxypropylmethyl cellulose, methacrylic acid, methyl methacrylate copolymer.
*Nebraska Cultures (Walnut Creek, California)

In a related embodiment, the invention provides SynBiotics which comprise probiotic mixtures consisting substantially of microbes whose viability has been partially attenuated, or probiotics consisting solely of non-viable microbes. The term “partially attenuated” includes mixtures consisting of 10%, 20%, 30%, 50% or more non-viable cells. The invention also provides SynBiotic formulations which comprise microbial membranes and/or cell walls that have been isolated and purified from killed microbes.

In addition to the above-described formulations, the present invention provides methods of using SynBiotics to treat the gastrointestinal ailments described herein. The amount of a SynBiotic administered will, of course, be dependent on the subject being treated, on the subject's weight, the severity of the affliction, and the manner of administration. A typical dosage for enteral administration is an amount from about 1 grams to about 5 grams per day. Determination of an effective amount is well within the capability of those skilled in the art.

As used herein, “effective amount,” or “therapeutically effective amount” refers to an amount of any of the compounds or formulations used in methods of the present invention that results in treatment of the medical condition, i.e., reduction in diarrhea or flatulence or any gastrointestinal pain. Alternatively, an “effective amount” can be determined by monitoring the presence of toxic microbes such as Clostridium difficile. In the context of the present invention, “prophylactically effective amount” refers to an amount of any of the present compounds that prevents the development or relapse of a medical condition. For example, a “prophylactically effective amount” is an amount that protects a subject from developing diarrhea.

For any compound or formulation used in a method of the invention, a therapeutically effective dose can be estimated initially from animal models (described supra), well known to those of skill in the art. Such information can be used to more accurately determine useful doses in humans. Initial dosages can also be estimated from in vitro or in vivo data.

Initial dosages can also be formulated by comparing the effectiveness of the compounds used in the methods of the present invention in model assays with the effectiveness of known compounds. For instance, initial dosages can be formulated by comparing the effectiveness of the compounds in model assays with the effectiveness of other compounds that have shown efficacy in treating the present conditions. In this method, an initial dosage can be obtained by multiplying the ratio of effective concentrations obtained in the model assay for the compounds used in methods of the present invention and the control compound by the effective dosage of the control compound. For example, if a compound useful in a present method is twice as effective in a model assay as a known compound (i.e., the EC50 of the compound is equal to one-half the EC50 of the known compound in the same assay), an initial effective dosage of the compound would be one-half the known dosage for the known compound. Using these initial guidelines one having ordinary skill in the art could readily determine an effective dosage in humans or other mammals.

Dosage amount and interval may be adjusted individually to provide levels of the active compound which are sufficient to maintain therapeutic effect. One having skill in the art will be able to optimize therapeutically effective local dosages without undue experimentation.

Combination Therapies. The SynBiotic formulations of the invention can be administered in combination with various therapies that are associated with gastrointestinal distress. Such therapies include, without limitation, radiation and chemotherapy for cancers, and antibiotic therapy for various microbial maladies. Such therapies tend to disrupt the composition and health of the intestine's normal fauna, leading to the undesirable proliferation of harmful bacteria and the accompanying painful symptoms described herein. Administration of the SynBiotic compositions described herein is useful for alleviating those symptoms.

### EXAMPLES

The following examples are offered to illustrate, but not to limit, the claimed invention.

### Example 1

### Preparation of a Soluble Stabilized Rice Bran Derivative

In order to generate the rice bran derivatives for use in the present invention, the rice bran is first stabilized, and then it is further separated into at least two fractions. These include, but are not limited to, a stabilized rice bran soluble derivative and a stabilized rice bran insoluble derivative. Preferably, the separation into the rice bran derivatives includes a non-chemical process i.e., an enzymatic process. In this process, partitioning or fractionation preferably proceeds as outlined hereinafter and described in U.S. Pat. No. 6,350,473, incorporated herein by reference.

The stabilized rice bran is made into about a 15% to about 35% slurry, preferably, a 20-25% slurry with potable water. An enzyme, which can include, but is not limited to, a dextranase, a maltase, an α-amylase, and various other carbohydrate cleaving enzymes, is added to the batch converting the starch to dextrins. The slurry is heated to about 150° F. to about 200° F. using, for instance, a steam injection cooker, a heat exchanger, or other heating method. The slurry is then pumped to a horizontal centrifuge wherein the insoluble fraction is separated. The insoluble fraction is collected and then dried on a belt dryer, and subsequently ground into a powder. This powder is the stabilized rice bran insoluble fraction. The aqueous material is pumped to a drum dryer and then dried. This dried aqueous portion produces the stabilized rice bran solubilized fraction.

The enzyme treated stabilized rice bran can be generated using the rice bran slurry as described above. As such, in another aspect, the present invention relates to the process for making an enzyme treated stabilized rice bran derivative, comprising: admixing stabilized rice bran with an aqueous solution to form about a 15% to about a 35% aqueous rice bran slurry, preferably a 20% to about a 30% aqueous rice bran slurry w/w; adding an enzyme to the aqueous rice bran slurry to convert starch to dextrin, thereby forming an enzyme treated slurry and then directly drying the enzyme treated slurry to form an enzyme treated stabilized rice bran derivative.

In a preferred embodiment of the foregoing process, after the enzyme is added to the slurry, the slurry is heated to about 100° F. to about 200° F. Preferably, the slurry is heated to about 150° F. to about 200° F. The slurry is then dried, wherein the drying is accomplished by a process such as belt drying, spray drying, drum drying and air drying. The drum drying process is preferred.

These stabilized rice bran derivatives are also available commercially from the NutraCea company of El Dorado Hills, Calif. Specifically, the insoluble derivative of stabilized rice bran is available as RiceMucil® Fiber Complex and the soluble derivative is available as RiSolubles®.

The stabilized rice bran derivatives can take a variety of forms. They can be a powder, a food, a food supplement, a medical food, a liquid, a beverage, an emulsion or mixture thereof. In addition, they can be incorporated into other edible materials. To incorporate the rice bran derivative into the diet of a mammal various options include, but are not limited to, simply sprinkling the derivative on another food substance (i.e., salad, bread, cereal, etc.) being a major ingredient in a multigrain ready to eat cereal, incorporating it into a baked product (breads, muffins, waffles, etc), pasta, healthy dessert and snacks (athletic bar, healthy drink, etc.) and high fiber foods.

Stabilized rice bran contains about 18-23% fat, about 23-35% dietary fiber, about 12-16% protein, about 8-36% total carbohydrate and many potent micro-components. Rice bran solubles contains about 15-40% fat, preferably 23-30% fat; about 0% to 25% dietary fiber, preferably about 0-20% dietary fiber; about 0% to 15% protein, preferably 6-9% protein and 25% to about 80% carbohydrates, preferably about 27-66% simple carbohydrate and is a water soluble fraction (see Table 1).

### Example 2

### Shelf Life of SynBiotics Formulation

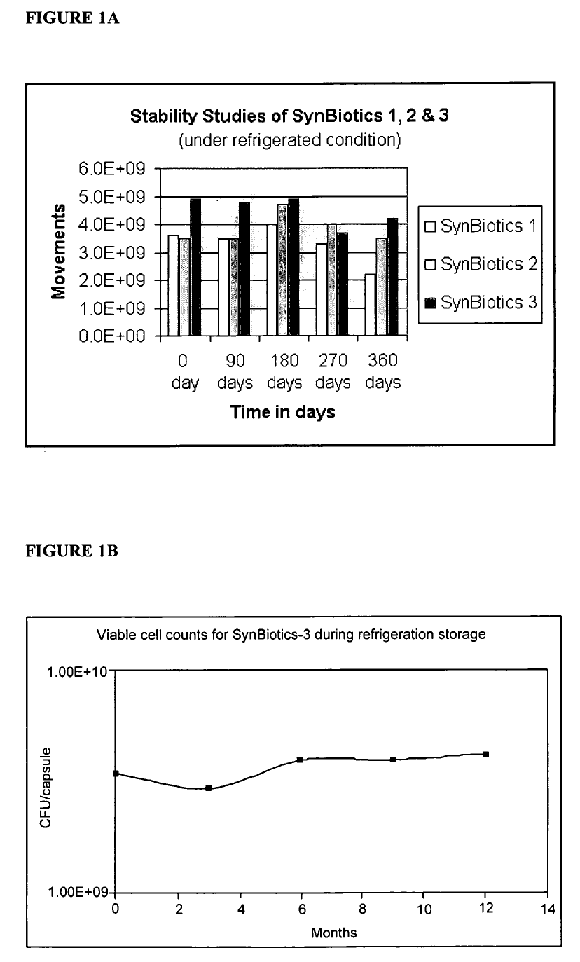

The shelf life of SynBiotics capsules under refrigerated (4° C.) conditions was determined by measuring the colony-forming units per capsule for SynBiotics 1, SynBiotics 2, and SynBiotics 3 encapsulated formulations. Measurements were taken approximately every three months, over the course of a year. The data are plotted in FIG. 1A.

A similar experiment was repeated with SynBiotics 3 capsules, measuring the total number of colony forming units per capsule over the course of a year. The results are shown in FIG. 1B. Together, the data show that the SynBiotic formulations have a shelf-life of at least one year.

### Example 3

### Home Study Protocol for SynBiotics Treatment

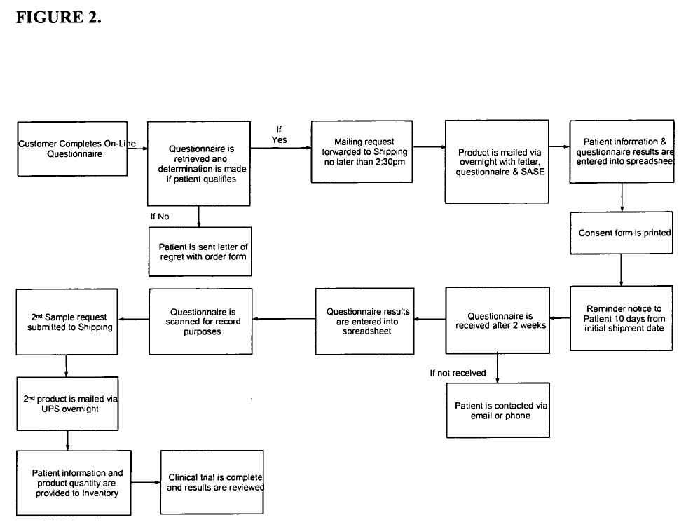

This Example describes an open-label study for people who have been diagnosed by their physician with irritable bowel syndrome and who wish to try a natural, easy to take, and effective product to treat their condition. The logic of the study is described in schematic form in FIG. 2.

The study is initiated with an on-line form presented to a candidate patient via a computer network, e.g., the World Wide Web. People who are interested in applying for the study complete the on-line questionnaire and consent form.

The applications are retrieved from the web-site and eligible patients are selected from the group of candidates based on their symptoms. If a patient is not eligible, a letter of regret is mailed along with an order form for purchasing a SynBiotic or other formulation of interest to the patient. If the candidate meets the eligibility requirements, a sample bottle of a SynBiotic formulation is express mailed, along with a congratulations letter, a Follow-up Questionnaire and a self-addressed stamped envelop (SASE). The participant's Consent to Participate is printed and filed for record purposes.

The participant's Information from the questionnaire is entered into the tracking spreadsheet. There are two worksheets: a demographics worksheet which maintains the participant's personal information and a questionnaire worksheet which keeps track of the answers to the questionnaires. Each participant will have two lines where the initial questionnaire answers and the answers to the two week Follow-Up Questionnaire are to be recorded.

The congratulations letter instructs the participant to keep the SynBiotics formulation refrigerated and provides recommendations for use, e.g., “take 2 capsules 2 times daily for 2 weeks.” The participant is reminded within 10 days of shipping to complete the follow-up portion Questionnaire. At the end of the two-week period they are asked again to complete the Follow-Up Questionnaire and return it using the SASE.

Once the Follow-Up Questionnaire answers are received from the participant, another product sample is mailed as a thank you to the participant for his or her participation in the study. An order form for additional SynBiotic formulations is also included with the sample.

The participant's information from the Follow-Up Questionnaire is entered into the tracking spreadsheet. The hard-copy Follow-up Questionnaire is also scanned and saved for historical records. The system just described can be carried out using Questionnaires that are solely computer-based, e.g., the answers to the Questionnaire may be entered by the participant using the participant's home computer and, after transmission over the Internet, the answers may be automatically recorded for future analysis, e.g., by the clinician in charge of the home trial.

Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

What is claimed is:

1. A synbiotic composition, said composition comprising
i) a probiotic composition, wherein said probiotic composition comprises a Lactobacillus species; and
ii) a prebiotic mixture, wherein said prebiotic mixture comprises at least one stabilized rice bran derivative, wherein said stabilized rice bran derivative comprises at least one natural antioxidant selected from the group consisting of a tocol, a phytosterol, γ-oryzanol and inositol hexaphosphate (IP6).

2. The composition of claim 1, wherein said at least one stabilized rice bran derivative is selected from the group consisting of: a water soluble fraction of stabilized rice bran; a water-insoluble fraction of stabilized rice bran; and a combination of water-soluble and water-insoluble fractions of stabilized rice bran.

3. The composition of claim 2, wherein said prebiotic mixture comprises at least one component selected from the group consisting of an oligosaccharide, a polysaccharide, and a fructo-oligosaccharide.

4. The composition of claim 3, further comprising a phytonutrient.

5. The composition of claim 2, wherein said prebiotic composition is hypoallergenic.

6. The composition of claim 2, wherein the amount of said prebiotic in said synbiotic composition is between 5% and 60% w/w.

7. The composition of claim 1, wherein said probiotic composition further comprises a Bifidobacteria species.

8. The composition of claim 7, wherein said Lactobacillus species comprises at least one species selected from the group of species consisting of L. caesi, L. acidophilus, L. plantarum, and L. rhamnosus.

9. The composition of claim 7, further comprising a yeast species.

10. The composition of claim 7, wherein said synbiotic composition is in a dosage form, and wherein said dosage form contains 10⁶ to 10¹⁰ viable colony forming units per dose.

11. The composition of claim 9, wherein said synbiotic composition comprises less than 20% prebiotics on a weight-percentage basis.

12. The composition of claim 11, wherein at least 40% w/w of said synbiotic composition consists of said yeast.

13. The composition of claim 7, wherein the composition comprises at least 20% prebiotics on a weight-percentage basis.

14. The composition of claim 7, wherein the composition comprises at least 40% prebiotics on a weight-percentage basis.

15. A vegetable capsule comprising the composition of claim 1.

16. The vegetable capsule of claim 15, wherein said vegetable capsule is enterically coated.

17. The composition of claim 1, wherein said composition is a liquid at room temperature.

18. The composition of claim 1, wherein said composition is in tablet form.

19. The composition of claim 1, wherein said composition is in powder form.

20. The composition of claim 1, wherein said composition is stable for at least one year when stored at temperatures between approximately 0 and 10 degrees Celsius.

21. A method for treating or preventing irritable bowel syndrome (IBS) and related bowel disorders, comprising the step of administering a therapeutically effective dose of the composition of claim 1 to a patient in need of such treatment.

22. The method of claim 21, wherein said patient has been diagnosed with an illness selected from the group consisting of: inflammatory bowel syndrome, Crohn's disease, ulcerative colitis, indeterminatal colitis, microscopic colitis, collagenous colitis, idiopathic inflammation of the small bowel, Clostridium difficile diarrhea, travelers' diarrhea, and antibiotic-induced diarrhea.

23. The method of claim 21, wherein said method enhances said subject's immune function.

24. The method of claim 21, wherein said method improves the subject's gut health.

25. The method of claim 21, wherein said method induces production of epithelial enzymes in said subject.

26. The method of claim 21, wherein said method induces the synthesis of vitamins in the intestines of said subject.

27. The method of claim 21, wherein said method results in a substantial reduction in the levels of toxins in said subject's gastrointestinal tract.

28. The method of claim 21, wherein said method induces apoptosis of cancer and precancerous cells in said subject.

29. The method of claim 21, wherein said method improves the overall gastrointestinal and colonic health of said subject.

30. The method of claim 21, wherein said method reduces bloating, abdominal distention or gas production in said subject.

31. The method of claim 21, wherein said method improves bowel regularity in said subject.

32. The method of claim 21, wherein said method facilitates healthy weight loss in said subject.

33. The method of claim 21, wherein said method treats or prevents harmful microbial and viral infections in said subject.

34. A method for carrying out a home study to determine the efficacy of a synbiotic composition, comprising the steps of:
providing an online questionnaire to candidate patients;
receiving a completed questionnaire from said candidate patients electronically;
determining from said completed questionnaire whether a candidate patient is eligible for said home study;
providing an eligible candidate with a test synbiotic formulation, instructions for administering said test synbiotic formulation over a predetermined time period, and a second questionnaire for said eligible candidate to self-record the effects of said synbiotic administration;
collecting the self-recorded data from said second questionnaire after said predetermined time period has passed; and
evaluating said self-recorded data.

35. The method of claim 34, wherein said test synbiotic formulation is the synbiotic composition of claim 1.
